(19) United States
(12) Patent Application Publication
(10) Pub. No.: US 2006/0039973 A1
(43) Pub. Date: Feb. 23, 2006

(54) Effervescent composition including water soluble dietary fiber

(76) Inventors: Mary Aldritt, Excelsior, MN (US); Fred Wehling, New Hope, MN (US); Robert E. Lee, Maple Grove, MN (US); Jason Kallestad, Minneapolis, MN (US)

Correspondence Address: ALLISON JOHNSON, P.A., LAKE CALHOUN EXECUTIVE CENTER, 3033 EXCELSIOR BLVD., SUITE 467, MINNEAPOLIS, MN, 55416, US

(21) Appl. No.: 11/207,245

(22) Filed: Aug. 19, 2005

Related U.S. Application Data
(60) Provisional application No. 60/602,920, filed on Aug. 19, 2004.

(51) Int. Cl. A61K 9/46; A61K 31/715

(52) U.S. Cl. 424/466; 514/54

(57) ABSTRACT

An effervescent composition that includes granulated water soluble dietary fiber comprising branched oligosaccharide including no greater than 10 sugar units, and an effervescent couple including acid and base.

### CROSS-REFERENCE TO RELATED APPLICATIONS

This application claims the benefit of U.S. Provisional Application No. 60/602,920, filed Aug. 19, 2005 and incorporated herein.

### BACKGROUND

The invention relates to forming an effervescent composition that includes water soluble dietary fiber.

Dietary fiber has been added to diets for a variety of purpose. Dietary fiber is known to function as a laxative when taken in relatively large amounts. Dietary fiber can also assist in the absorption of vitamins and minerals and can decrease cholesterol. To achieve this effect without achieving the laxative effect it is often desirable for an individual to intake a lesser amount of dietary fiber.

Laxative formulations that include dietary fiber often include water insoluble forms of dietary fiber such as cellulose, psyllium husk, bran, and wheat germ. Dietary fiber is sold in a variety of forms including powdered drink mixes. Various dietary fiber powdered drink mixes are available under the METAMUCIL series of trade designations from Procter & Gamble (Cincinnati, Ohio).

Powdered drink mixes are typically added to a glass of water and then consumed by the individual as a liquid beverage. One drawback of powdered drink mixes that include dietary fiber is that the mix tends to gelatinize when added to water or before the entire beverage is consumed. Gelatinous mixtures are difficult to swallow especially for children and the elderly and can cause the gag reflex to occur.

Effervescent compositions are a useful dosage form for delivering active agents because they can be packaged in discreet and controlled quantities and tend to be easier to swallow relative to a tablet or capsule. Effervescent compositions are added to water where they disintegrate and preferably dissolve to form a beverage. Consumers tend to prefer effervescent compositions that dissolve in water due to ease of drinking. Effervescent compositions that coagulate can be aesthetically undesirable to the consumer and difficult to swallow.

As a dosage form, effervescent compositions preferably dissolve rapidly to a relatively clear solution, and form a liquid composition that can be swallowed easily and that exhibits an aesthetically acceptable appearance to the consumer. However, when preparing a new effervescent composition with one or more new compounds it is often difficult to achieve the preferred properties of an effervescent composition. There are further difficulties associated with formulating effervescent compositions for tableting. It is often very difficult, for example, to achieve a formulation that can be manufactured in a high speed tableting operation to produce tablets that release from the tablet press with an acceptable appearance, exhibit a hardness suitable for shipping, maintain their integrity until use, exhibit a suitable dissolution profile, and the other properties desired of an effervescent dosage form.

### SUMMARY

In one aspect, the invention features an effervescent composition that includes granulated water soluble dietary fiber that includes branched oligosaccharide that includes at least three and no greater than ten sugar units, and an effervescent couple that includes acid and base, the composition disintegrating in 22° C. water to a clear composition in less than six minutes. In one embodiment, the dietary fiber has an average particle size no greater than 1000 μm. In another embodiment, the dietary fiber has an average particle size no greater than 700 μm.

In some embodiments, the composition, when added to room temperature water, disintegrates to a clear composition in less than 4 minutes.

In other embodiments, the composition, when added to room temperature water, forms an aqueous composition that is free of gelling and remains free of gelling for at least five minutes.

In other embodiments, the composition further includes sorbitol.

In other embodiments, the oligosaccharide includes fructo-oligosaccharide. In one embodiment, the composition includes from 25% by weight to 55% by weight the fructo-oligosaccharide. In another embodiment, the composition includes from 30% by weight to 50% by weight the fructo-oligosaccharide. In some embodiments, the composition includes from 35% by weight to 50% by weight the fructo-oligosaccharide.

In one embodiment, the composition includes at least 2.5 g the dietary fiber.

In other embodiments, the composition further includes polyethylene glycol and sodium benzoate. In another embodiment, the composition further includes sorbitol, polyethylene glycol, and sodium benzoate. In other embodiments, the composition further includes from 1% by weight to 25% by weight sorbitol, from 0.1% by weight to 10% by weight polyethylene glycol, and from 0.1% by weight to 10% by weight sodium benzoate. In another embodiment, the composition further includes from 50 mg to 1250 mg sorbitol, from 5 mg to 500 mg polyethylene glycol, and from 5 mg to 500 mg sodium benzoate.

In another embodiment, the composition includes at least 2.5 g dietary fiber, and further includes from 50 mg to 1250 mg sorbitol, from 5 mg to 500 mg polyethylene glycol, and from 5 mg to 500 mg sodium benzoate.

In other embodiments, the composition further includes from 50 mg to 1250 mg by weight sorbitol, from 50 mg to 1500 mg sodium bicarbonate, and from 50 mg to 1500 mg potassium bicarbonate.

In some embodiments, the composition further includes sucralose.

In one embodiment, the effervescent composition is an effervescent tablet that includes at least 2.5 g granulated water soluble dietary fiber that includes branched oligosaccharide that includes at least 3 and no greater than 10 sugar units, and an effervescent couple that includes acid and base, the tablet exhibiting a hardness of at least 3 Kp and disintegrating in 22° C. water in less than six minutes. In some embodiments, the tablet has a hardness of at least 6 Kp.

In another aspect, the invention features a packaged effervescent dosage form that includes an airtight sealed foil pouch, and an effervescent composition disposed in the pouch, the effervescent composition includes an effervescent couple that includes acid and base, at least 2 g dietary fiber, the composition disintegrating in 22° C. water in less than six minutes.

In some embodiments, the packaged dosage form, when stored for 5 days at 45° C., is free of puffing. In some embodiments, the packaged dosage form, when stored for 5 days at 40° C. and 75% relative humidity, is free of puffing.

In another aspect, the invention features a method of making an effervescent composition that includes dietary fiber, the method includes drying the dietary fiber at a temperature less than 50° C., and combining the dried dietary fiber and an effervescent agent with mixing.

The effervescent composition exhibits good compressibility and can be tableted using a high speed tableting operation into tablets and powders that include a relative high dietary fiber loading, exhibit a good dissolution rate, and disintegrate to a relatively clear solution.

Other features and advantages will be apparent from the following description of the preferred embodiments and from the claims.

### GLOSSARY

In reference to the invention, these terms have the meanings set forth below:

Effervescent means the ability to rapidly give off gas bubbles when contacted with water.

### DETAILED DESCRIPTION

The effervescent composition includes dietary fiber that includes branched oligosaccharide having from at least 3 to no greater than about 10 sugar units and an effervescent agent. The effervescent composition is water soluble such that it disintegrates (i.e., dissolves, disperses, disintegrates or a combination thereof) in water. The effervescent composition evolves bubbles carbon dioxide gas as it disintegrates in water. Preferably the effervescent composition forms at least a substantially uniform dispersion, more preferably dissolves to a clear solution, when placed in a sufficient amount of water. The uniformity and clarity of the composition is determined by viewing with the naked eye. The effervescent composition preferably dissolves in water to form a solution that is free of gelling and remains gel free for at least five hours.

Preferably the effervescent composition dissolves in excess water at room temperature (about 22° C.) in less than four minutes, or even less than three minutes. The composition preferably is self-mixing, i.e., when excess water is added to the effervescent composition, the effervescent composition will dissolve on its own without mixing or stirring from another source. Of course the composition can be stirred to assist and increase the rate of dissolution. The resulting aqueous composition preferably has a pH from 3 to 5, is palatable, and can be easily swallowed.

The effervescent composition is preferably a homogeneous mixture of each of the ingredients present in the composition. The homogenous mixture is granular and passes through a number 12 sieve. The effervescent composition is preferably provided in a useful dosage form including, e.g., tablets weighing about 3 g, about 5 g, or even about 6 g, and sachets filled with a free flowing granulation in amounts of about 3 g, about 5 g, or even about 7 g.

The dietary fiber of the effervescent composition is water soluble and is preferably granulated. The dietary fiber preferably dissolves to a clear solution (as determined by the naked eye) in water. The granulated dietary fiber preferably has an average particle size of no greater than 1000 μm, or even no greater than 700 μm. The dietary fiber preferably is of a kind and present in an amount sufficient to promote the growth of beneficial Bifidobacteria in the lower gut and increase the absorption of dietary calcium. Useful dietary fibers provide good compressibility for forming tablets, and exhibit a good dissolution rate. Preferred dietary fiber has a bulk density of greater than 30 g/ml, greater than 40 g/ml, greater than 50 g/ml, greater than 60 g/ml, or even greater that 80 g/ml. Suitable dietary fibers include water soluble branched oligosaccharides having at least about 3, no greater than 10, no greater than about 8, from about 3 to about 10, or even from about 8 to 10 sugar units. An example of a useful source of branched oligosaccharide dietary fiber is chicory fructo-oligosaccharide, i.e., fructo-oligosaccharide derived from chicory roots. Preferred commercially available water soluble dietary fibers are available under the OLIGGO*FIBER series of trade designations from Cargill Health and Food Technologies (Wayzata, Minn.) and include OLIGGO*FIBER INSTANT and OLIGGO*FIBER INULIN. Other suitable water soluble dietary fibers are available under the FIBERAID trade designation from Larex, Inc (White Bear Lake, Minn.).

The effervescent composition preferably includes at least about 25% by weight, at least about 30% by weight, at least about 35% by weight, no greater than about 55% by weight, no greater than about 50% by weight, no greater than about 45% by weight dietary fiber. The dose of dietary fiber provided by the effervescent composition preferably is at least 1 g, at least about 2 g, at least about 3 g, no greater than about 6 g, or even from about 1 g to about 3 g dietary fiber. A useful dose regime for an individual includes two doses of fiber a day for a total of at least about 5 g, or even no greater than about 15 g dietary fiber per day.

The dietary fiber is preferably dried before being incorporated into the effervescent formulation. Useful drying methods include drying the dietary fiber at a temperature less than 50° C., or even no greater than about 45° C. for at least about 16 hours, or even at least about 24 hours.

The effervescent agent includes an effervescent couple that includes an acid and a base. The effervescent couple is activated when contacted with water, e.g., when the powder or tablet is placed in a glass of water. The water liberates the acid and base and enables the acid and base to react with each other to produce carbon dioxide gas, which imparts carbonation to the aqueous composition. Examples of useful acids include citric acid, ascorbic acid, aspartic acid, malic acid, adipic acid, tartaric acid, fumaric acid, succinic acid, sodium acid pyrophosophate, lactic acid, hexamic acid, amino acids, and acid salts and acid anhydrides thereof, and mixtures thereof. Examples of useful acid anhydrides include citraconic anhydride, glucono-D-lactone, and succinic anhydride. Examples of useful acid salts include potassium bitartrate, acid citrate salts, sodium dihydrogen phosphate, disodium dihydrogen phosphate, sodium acid sulfite, and combinations thereof. Preferably acid is present in the composition in an amount of from 10% by weight to about 60% by weight, from about 15% by weight to about 50% by weight, or even from about 25% by weight to about 40% by weight. Useful dosage forms include from about 500 mg to about 3000 mg, from about 750 mg to about 2500 mg, or even from about 1250 mg to about 2000 mg of the acid component of the effervescent couple.

The base preferably is capable of generating carbon dioxide. Examples of suitable carbonate bases include sodium bicarbonate, sodium carbonate, sodium sesquicarbonate, potassium carbonate, potassium bicarbonate, calcium carbonate, magnesium carbonate, magnesium oxide, sodium glycine carbonate, L-lysine carbonate, arginine carbonate, zinc carbonate, zinc oxide, amino acid carbonates, and mixtures thereof. The composition preferably includes base in an amount of from 10% by weight to about 60% by weight, from about 15% by weight to about 50% by weight, or even from about 25% by weight to about 40% by weight. Useful dosage forms include from about 500 mg to about 3000 mg, from about 750 mg to about 2500 mg, or even from about 1250 mg to about 2000 mg of the base component of the effervescent couple.

The effervescent composition can optionally include a variety of additional active agents including, e.g., vitamins, amino acids, pharmaceutical agents, minerals, dietary supplements, and combinations thereof. Suitable vitamins include, e.g., ascorbic acid (vitamin C), aspartic acid, thiamin, riboflavin, nicotinic acid, pantothenic acid, pyridoxine, biotin, folic acid, niacin, vitamin B12, lipoic acid, vitamin A, vitamin D, vitamin E and vitamin K and coenzymes thereof, choline, carnitine, and alpha, beta, and gamma carotenes. Examples of coenzymes include thiamine pyrophosphates, flavin mononucleotide, flavin adenine dinucleotide, nicotinamide adenine dinucleotide, nicotinamide adenine dinucleotide phosphate coenzyme A pyridoxal phosphate, biocytin, tetrahydrofolic acid, coenzyme B12, lipoyllysine, 11-cis-retinal, and 1,25-dihydroxycholecalciferol and mixtures.

Suitable amino acids include, e.g., L-tyrosine, isoleucine, ornithine, glutamine, phenylalanine, leucine, lysine, methionine, threonine, taurine, tryptophan, valine, alanine, glycine, arginine, histidine, cysteine, asparagine, proline and serine, and mixtures thereof.

Examples of minerals include iron, zinc, selenium, copper, iodine, phosphorus, chromium and mixtures thereof.

Suitable dietary supplements include, e.g., bee pollen, bran, wheat germ, kelp, cod liver oil, ginseng, and fish oils, amino-acids, proteins, vitamins, minerals alpha-glycerylphosphorylcholine, acetyl-L-carnitine and salts thereof, docosahexaenoic acid, glucosamine, chondroitin, methylsulfonylmethane, and mixtures thereof.

The composition can also include other ingredients including, e.g., flavor agents, fillers, surfactants (e.g., polysorbate 80 and sodium lauryl sulfate), color agents including, e.g., dyes and pigments, and sweeteners.

Useful flavor agents include natural and synthetic flavoring sources including, e.g., volatile oils, synthetic flavor oils, flavoring aromatics, oils, liquids, oleoresins and extracts derived from plants, leaves, flowers, fruits, stems and combinations thereof. Useful flavor agents include, e.g., citric oils, e.g., lemon, orange, grape, lime and grapefruit, fruit essences including, e.g., apple, pear, peach, grape, strawberry, raspberry, cherry, plum, pineapple, apricot, and other fruit flavors. Other useful flavor agents include, e.g., aldehydes and esters (e.g., benzaldehyde (cherry, almond)), citral, i.e., alpha-citral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), 2-dodedenal (citrus, mandarin) and mixtures thereof.

Useful color agents include, e.g., food, drug and cosmetic (FD&C) colors including, e.g., dyes, lakes, and certain natural and derived colorants. Useful lakes include dyes absorbed on aluminum hydroxide and other suitable carriers.

Useful sweetening agents include stevia, sugars such as sucrose, glucose, invert sugar, fructose, ribose, tagalose, sucralose, malitol, erythritol, xylitol, and mixtures thereof, saccharin and its various salts (e.g., sodium and calcium salt of saccharin), cyclamic acid and its various salts, dipeptide sweeteners (e.g., aspartame), acesulfame potassium, dihydrochalcone, glycyrrhizin, and sugar alcohols including, e.g., sorbitol, sorbitol syrup, mannitol and xylitol, and combinations thereof.

The effervescent composition can be in a variety of forms including, e.g., powder (e.g., a free flowing granulation), tablet, capsule, pellet and composite. Preferred effervescent tablets have a hardness of at least 3 kilopounds (Kp), preferably at least 6 Kp, from about 6 Kp to about 10 Kp, or even from about 6 Kp to about 8 Kp, as measured on a standard hardness tester (e.g., tablet hardness testers available from Dr. Schleuniger Pharmatron, Inc., Germany).

When in the form of a tablet, the composition preferably includes binder, lubricant, and combinations thereof. Examples of suitable binders include, e.g., starches, natural gums, cellulose gums, microcrystalline cellulose, methylcellulose, cellulose ethers, sodium carboxymethylcellulose, ethylcellulose, gelatin, dextrose, lactose, sucrose, sorbitol, mannitol, polyethylene glycol, polyvinylpyrrolidone, pectins, alginates, polyacrylamides, polyvinyloxoazolidone, polyvinylalcohols and mixtures thereof.

Where present, the composition includes a sufficient amount of binder to assist in holding the components of the composition together in the form of a tablet. When present, the composition preferably includes binder in an amount of from 10% by weight to about 60% by weight, from about 15% by weight to about 50% by weight, or even from about 25% by weight to about 40% by weight. Useful dosage forms include from about 500 mg to about 3000 mg, from about 750 mg to about 2500 mg, or even from about 1250 mg to about 2000 mg binder.

Various lubricants are suitable for use in the composition including water dispersible, water soluble, water insoluble lubricants and combinations thereof. Preferred lubricants are water soluble. Examples of useful water soluble lubricants include sodium benzoate, polyethylene glycol, L-leucine, adipic acid, and combinations thereof. The composition can also include water insoluble lubricants including, e.g., stearates (e.g., magnesium stearate, calcium stearate and zinc stearate), oils (e.g., mineral oil, hydrogenated and partially hydrogenated vegetable oils, and cotton seed oil) and combinations thereof. Other water insoluble lubricants include, e.g., animal fats, polyoxyethylene monostearate, talc, and combinations thereof.

The composition preferably includes a sufficient amount of lubricant to enable the composition to be formed into tablets and released from a high speed tableting press in the form of a tablet. When present, the composition preferably includes lubricant in an amount of from 0.1% by weight to about 15% by weight, from about 1% by weight to about 12% by weight, from about 2% by weight to about 10% by weight, or even from about 3% by weight to about 8% by weight. Useful dosage forms include at least 5 mg, from about 50 mg to about 750 mg, from about 50 mg to about 600 mg, from about 100 mg to about 500 mg, or even from about 150 mg to about 400 mg lubricant.

The effervescent composition is preferably stored in a moisture-proof package including, e.g., sealed metal foil pouches, blister packs, and desiccant capped tubes. The composition can be administered by dissolving the composition in excess water, e.g., an eight ounce glass of tap water, to form an aqueous solution and ingested. After addition of the effervescent composition to an aqueous liquid, the composition optionally can be stirred to facilitate dispersion and/or dissolution in the aqueous liquid.

The invention will now be described by way of the following examples. All of the weight units are in mg unless otherwise specified.

### EXAMPLES

### Example 1

An effervescent composition in the form of a granulated powder was prepared by combining sieved citric acid, sodium bicarbonate, potassium bicarbonate, sodium carbonate, sorbitol, PEG 3350 polyethylene glycol, sodium benzoate, OLIGGO FIBER INSTANT fructo-oligosaccharide having an average particle size less than 700 μm (Cargill Health and Food Technologies, Wayzata, Minn.), FIBERAID larch arabinogalactan dietary fiber having a bulk density of from 30 g/ml to 40 g/ml (Larex, Inc., White Bear Lake, Minn.), sucralose, and 7-Up flavoring, in the amounts in grams (g) set forth in Table 1 below, in a blender with mixing.

The composition of Example 1 was then tableted to an average tablet hardness of 6.5 Kp. A tablet was removed from the batch and placed in 200 mL of tap water. The tablet completely dissolved in three minutes to a clear solution with no precipitate. The tablets were then individually sealed in foil pouches.

Four sealed tablets were stored at 40° C. and 75% relative humidity. After five days of storage the pouches were observed to exhibit minimal puffing. After four weeks of storage the pouches were observed to exhibit minimal puffing and the tablets exhibited a good appearance.

Four sealed tablets were stored at 45° C. After five days of storage the pouches were observed to exhibit mild puffing.

### Example 2

An effervescent composition in the form of a granulated powder was prepared by combining sieved citric acid, sodium bicarbonate, potassium bicarbonate, sodium carbonate, anhydrose dextrose, OLIGGO FIBER INSTANT fructo-oligosaccharide (Cargill Health and Food Technologies), FIBERAID larch arabinogalactan dietary fiber (Larex, Inc.), sucralose, and 7-Up flavoring in the amounts (g) set forth in Table 1 below, in a blender with mixing. The dietary fiber was dried for 16 hours at 45° C. prior to being added to the formulation.

A 6.77 g sample of the powder was dissolved in 200 mL room temperature tap water. The powder dissolved about 1.5 minutes. Some particulate was seen on the bottom and side wall of the vessel.

Samples of the powder of Example 2, each weighing 6.77 g, were sealed in foil pouches to form a sachet.

Four foil sachets were stored at 40° C. and 75% relative humidity. After five days little or no puffing was observed. The contents of the sachet were observed to be a white free flowing powder that was free of clumping. After four weeks little or no puffing was observed: One sachet was opened and the contents of the sachet was observed to be a white free flowing powder that was free of clumping.

Four foil sachets were stored at 45° C. After five days little or no puffing was observed. One sachet was opened and the contents of the sachet were observed to be a white free flowing powder that was free of clumping.

### Example 3

The composition of Example 3 was prepared according to the method described in Example 1 with the exception that ingredients and the amounts were as set forth in Table 1.

The composition of Example 3 was tableted with a one inch Teflon tool on a CADMAC press to an average tablet hardness of 7 Kp. The tablets all capped. No picking was observed.

A tablet was removed from the batch and placed in 200 mL of tap water. The tablet exhibited weak effervescence and completely dissolved to a clear, colorless solution in about four minutes.

The tablets were individually sealed in foil pouches. Four of the foil sealed tablets were stored at 45° C. After six days the sealed foil packages were removed from the oven and observed to be free of puffing.

### Example 4

The composition of Example 4 was prepared according to the method described in Example 1 with the exception that ingredients and the amounts were as set forth in Table 1. The composition of Example 4 was tableted to an average weight of 5.5 g and an average tablet hardness of 6.1 Kp. The tablets were free of capping and picking. Some die wall etching was observed. A tablet was removed from the batch and was placed in 200 mL of tap water. The tablet completely dissolved in three minutes 45 seconds to a clear solution with no precipitate.

### Example 5

The composition of Example 5 was prepared substantially according to the method described in Example 1 with the exception that after an initial blend of some of the ingredients a portion of that blend was combined with additional ingredients to achieve a composition including the ingredients in the amounts set forth in Table 1. The composition of Example 5 was then tableted on a one inch Teflon coated tool. The composition tableted well and was capable of being compressed to 15 Kp without capping. No picking or die wall etching was observed. The composition was tabletted to an average hardness of 5.8 Kp and an average tablet weight of about 7 g.

The tablet was placed in 200 mL of room temperature water and dissolved in about ten minutes. The tablet was placed in 200 mL of hot water and dissolved in about 30 to 45 seconds.

### Example 6

The composition of Example 6 was prepared as described in Example 1 with the exception that fine citric acid was used (Cargill Foods reported as having a particle size characterized by a maximum of 1% on U.S. No. 30 sieve and maximum of 10% through U.S. No. 100 sieve). The composition was tableted on a one inch Teflon coated tool to an average tablet weight of 5.5 g and an average tablet hardness of 6.5 Kp. The tablets were free of capping, picking and die wall etching.

A tablet from the batch was placed in 200 mL of room temperature water and was observed to dissolve in four minutes, 15 seconds.

TABLE 1

| | Example | | | | | |
|---|---|---|---|---|---|---|
| Ingredient (g) | 1 | 2 | 3 | 4 | 5 | 6 |
| Citric Acid | 540 | 135 | 405 | 1350 | 202.5 | 355 |
| Sodium | 194 | 48.5 | 145 | 485 | 72.8 | 121.25 |
| Bicarbonate | | | | | | |
| Potassium | 204.8 | 51.2 | 153 | 512 | 76.8 | 128 |
| bicarbonate | | | | | | |
| Sodium | 40 | 10 | 30 | 100 | 15 | 25 |
| carbonate | | | | | | |
| Sorbitol | 320 | 0 | 90 | 600 | 180 | 150 |
| Anhydrose | 0 | 100 | 0 | 0 | 0 | 0 |
| Dextrose | | | | | | |
| PEG 3350 | 80 | 0 | 60 | 200 | 30 | 50 |
| Sodium | 50 | 0 | 37.5 | 125 | 21 | 35 |
| Benzoate | | | | | | |
| Dietary | 320 | 160 | 450 | 2000 | 450 | 500 |
| Fiber¹ | | | | | | |
| Dietary | 320 | 160 | 450 | 0 | 0 | 0 |
| Fiber² | | | | | | |
| Sucralose | 8 | 2 | 6 | 20 | 3.0 | 5.5 |
| Flavoring | 40 | 10 | 30 | 100 | 15 | 25 |

¹= OLIGGO FIBER INSTANT fructo-oligosaccharide (Cargill Health and Food Technologies, Wayzata, Minnesota).
²= FIBERAID larch arabinogalactan dietary fiber (Larex, Inc., White Bear Lake, Minnesota).

Other embodiments are within the claims.

What is claimed is:

1. An effervescent composition comprising:
a) granulated water soluble dietary fiber comprising branched oligosaccharide comprising at least three and no greater than ten sugar units; and
b) an effervescent couple comprising acid and base,
the composition disintegrating in 22° C. water to a clear composition in less than six minutes.

2. The effervescent composition of claim 1, wherein the dietary fiber has an average particle size no greater than 1000 μm.

3. The effervescent composition of claim 1, wherein the dietary fiber has an average particle size no greater than 700 μm.

4. The effervescent composition of claim 1, wherein the composition, when added to room temperature water, disintegrates to a clear composition in less than 4 minutes.

5. The effervescent composition of claim 1, wherein the composition, when added to room temperature water, forms an aqueous composition that is free of gelling and remains free of gelling for at least five minutes.

6. The effervescent composition of claim 1, wherein the composition further comprises sorbitol.

7. The effervescent composition of claim 1, wherein the oligosaccharide comprises fructo-oligosaccharide.

8. The effervescent composition of claim 1, wherein the composition comprises from 25% by weight to 55% by weight fructo-oligosaccharide.

9. The effervescent composition of claim 1, comprising from 30% by weight to 50% by weight the fructo-oligosaccharide.

10. The effervescent composition of claim 1, comprising from 35% by weight to 50% by weight the fructo-oligosaccharide.

11. The effervescent composition of claim 1, comprising at least 2.5 g dietary fiber.

12. The effervescent composition of claim 1, further comprising polyethylene glycol and sodium benzoate.

13. The effervescent composition of claim 1, further comprising sorbitol, polyethylene glycol, and sodium benzoate.

14. The effervescent composition of claim 1, further comprising
from 1% by weight to 25% by weight sorbitol,
from 0.1% by weight to 10% by weight polyethylene glycol, and
from 0.1% by weight to 10% by weight sodium benzoate.

15. The effervescent composition of claim 1, further comprising
from 50 mg to 1250 mg sorbitol,
from 5 mg to 500 mg polyethylene glycol, and
from 5 mg to 500 mg sodium benzoate.

16. The effervescent composition of claim 1, comprising at least 2.5 g dietary fiber, and further comprising
from 50 mg to 1250 mg sorbitol,
from 5 mg to 500 mg polyethylene glycol, and
from 5 mg to 500 mg sodium benzoate.

17. The effervescent composition of claim 1 further comprising
from 50 mg to 1250 mg by weight sorbitol,
from 50 mg to 1500 mg sodium bicarbonate, and
from 50 mg to 1500 mg potassium bicarbonate.

18. The effervescent composition of claim 1 further comprising sucralose.

19. An effervescent tablet comprising
at least 2.5 g granulated water soluble dietary fiber comprising branched oligosaccharide comprising at least three and no greater than ten sugar units, and
an effervescent couple comprising acid and base,
the tablet exhibiting a hardness of at least 3 Kp and disintegrating in 22° C. water in less than six minutes.

20. The tablet of claim 19, wherein the tablet has a hardness of at least 6 Kp.

21. A package comprising:
an airtight sealed foil pouch, and
an effervescent composition disposed in the pouch, the effervescent composition comprising
an effervescent couple comprising acid and base, and
at least 2 g dietary fiber,
the composition disintegrating in 22° C. water in less than six minutes.

22. The package of claim 21, wherein the package is free of puffing after storage for 5 days at 45° C.

23. The package of claim 21, wherein the package is free of puffing after storage for 5 days at 40° C. and 75% relative humidity.

24. A method of making an effervescent composition comprising dietary fiber, the method comprising
drying the dietary fiber at a temperature less than 50° C.; and
combining the dried dietary fiber and an effervescent agent with mixing.

25. An effervescent composition comprising:
water soluble dietary fiber;
an effervescent couple comprising acid and base,
from 50 mg to 1250 mg sorbitol;
from 5 mg to 500 mg polyethylene glycol;
from 5 mg to 500 mg sodium benzoate;
said composition disintegrating in 22° C. water to a clear composition in less than six minutes.
